(19) United States
(12) Patent Application Publication
(10) Pub. No.: US 2014/0037552 A1
(43) Pub. Date: Feb. 6, 2014

(54) PRUSSIAN BLUE BASED NANOPARTICLE AS MULTIMODAL IMAGING CONTRAST MATERIAL

(71) Applicants: Domokos Máthé, Budapest (HU); Krisztián Szigeti, Budapest (HU)

(72) Inventors: Domokos Máthé, Budapest (HU); Krisztián Szigeti, Budapest (HU)

(21) Appl. No.: 13/985,254

(22) PCT Filed: Feb. 15, 2012
(86) PCT No.: PCT/HU12/00010

§ 371 Date: Oct. 18, 2013

(30) Foreign Application Priority Data
Feb. 15, 2011 (HU) P1100080

(51) Int. Cl. A61K 49/00; A61K 49/04

(52) U.S. Cl. CPC A61K 49/0002; A61K 49/04
USPC 424/9.4; 424/9.1; 427/2.11

(57) ABSTRACT

The invention relates to a Prussian Blue based nanoparticle comprising a Prussian Blue based metal core doped with one or more metal isotope and an organic biocompatible coating. The invention relates furthermore to a process for the preparation of said nanoparticle, and the use thereof as imaging contrast material or in the therapy.

### FIELD OF THE INVENTION

This invention relates to Prussian Blue based nanoparticles as multimodal imaging contrast material. The invention relates furthermore to a process for the preparation of said nanoparticles, and the use thereof.

### BACKGROUND OF THE INVENTION

The present invention relates to the fine and superfine particle of Prussian Blue type complex with nanometer-scale size. Metal-complexes consisting of particular metals and particular coordination molecules show various properties depending on the combination of the kind of metal and kind of coordination molecules. In the present invention the crystal of Prussian Blue complexes includes certain modifications, such as substitution or defects of the hexacyano-metallic group, the transition metals forming Prussian Blue-like structures and intercalation of various ions in the crystals, and water.

Iron-containing nanoparticles have found widespread biomedical applications such as magnetic-field guided drug delivery, hyperthermia for cancer treatment, and medical imaging (e.g. magnetic resonance imaging (MRI)). Most frequently iron oxide based nanoparticles are applied. There are many forms of iron oxide nanoparticles used in academic and clinical applications which can be divided into three categories: oral Superparamagnetic Iron Oxide (SPIO) (size: 3500 nm-200 nm), standard SPIO (SSPIO) (size: 200-50 nm); and ultrasmall SPIO (USPIO) (size: <50 nm). The SPIO nanoparticles usually are composed of a water insoluble crystalline magnetic core, the mean core diameter ranging from 4 to 100 nm. This crystalline core is often surrounded by different surface coatings. Polymeric coatings represent the most common class of surface coatings used to improve the bio-compatibility and stability of iron containing nanoparticles. Some examples include dextran, carboxymethylated dextran, polyvinyl alcohol (PVA), starches, chitosan, etc.

WO 96/04017 discloses an iron-containing nanoparticle with modular structure (core, polymer coat and targeting polymer coat). The disclosure focuses on MRI applications of iron- and other metal-containing nanoparticles. WO 96/03653 discloses a superparamagnetic nanoparticle built up from one or aggregated one-domain nanoparticle containing iron-oxide and iron-hydroxide, and having a particle size in the 3-50 nm range.

WO 2009/136764 discloses a dual modality PET/MRI contrast material the core of which is built of iron-oxide or metallic alloy.

Prussian Blue is a dark blue micro-crystalline pigment, one of the first synthetic dyes. The Prussian Blue dye is a common histopathology (ex vivo) stain, used to detect the presence of iron in biopsy specimens, such as in bone marrow samples. Iron deposits in tissue are visualized as blue or purple deposits of Prussian Blue dye formed by a specific histochemical reaction in situ.

In the last decade some research groups produced Prussian Blue nanoparticles which were tested then as potential MRI contrast agents and drug delivery systems. In order for Prussian Blue to be successfully utilized as drug delivery agents they must be capable of crossing the plasma membrane. To study intracellular uptake of the Prussian Blue particles the surfaces were functionalized with small molecules, or an anti-tumor agent for instance doxorubicin. The functionalized Prussian Blue particles with both MRI contrast and drug delivery capabilities may become powerful dual agents for simultaneous cancer treatment and assessment of treatment effectiveness.

Finding scalable manufacturing processes of nanoparticles is very important for practical application of Prussian Blue and its analogues. There have been a few reports of the method to produce such nanoparticles. The majority of those, however, do not suit for inexpensive and simple mass production because of the complexity of the synthesizing process (Yamada, JACS 126 (2004) p9482). U.S. Pat. No. 7,678,188 and Shokouhimehr, Inorg Chem Comm 13 (2010) 58-61 disclose an easy and efficient method to mass production of Prussian Blue nanoparticles but have not presented any other multimodal imaging applications except MRI.

The cytotoxicity of Prussian Blue nanoparticles is a more crucial problem compared with other nanoparticles by reason of cyanide content, therefore it was assessed and the results show that the cyanide groups do not pose any toxicity problem. In addition, the nanoparticle is very stable hereby no toxic effects of freely available cyanide groups can be expected.

Prussian Blue's ability to incorporate mono-cations makes it useful as a sequestering agent for certain heavy metal poisons. The U.S. Food and Drug Administration (FDA) has determined that Prussian Blue dosed orally is safe and effective in gastrointestinal decontamination therapy of certain metal ions and isotopes such as ¹³⁷Cs (Hoffman R S et al.: “Comparative efficacy of thallium adsorption by activated charcoal, Prussian Blue, and sodium polystyrene sulfonate” J. Toxicol. Clin. Toxicol. 37(7), 833-837 (1999)).

The present invention relates to metal intercalation ability of Prussian Blue which allows various applications in multimodal imaging and in the therapy.

### SUMMARY OF THE INVENTION

The present invention relates to Prussian Blue based nanoparticles that have a modular structure, and the use thereof for diagnostic and therapeutic purposes. The nanoparticles according to the invention comprise a Prussian Blue based metal core doped with metal isotope(s) and an organic biocompatible coating. The nanoparticles according to the invention can be used as contrast material for detection with different imaging modalities such as MRI, X-ray Computed Tomography (CT), Single Photon Emission Computed Tomography (SPECT), Positron Emission Tomography (PET) or optical imaging (OI) methods during clinical, in vivo, or in vitro studies. Furthermore, the nanoparticles according to the invention can be used for therapeutic purposes.

### BRIEF DESCRIPTION OF DRAWINGS

FIG. 1 shows a Prussian Blue or analogue nanoparticle with primary metal coat and optionally secondary associated biomaterials.

FIG. 2 shows a Prussian Blue or analogue nanoparticle with mixed position metal coat and optionally secondary associated biomaterials.

FIG. 3 shows a Prussian Blue or analogue nanoparticle with mixed position metal coat and optionally secondary associated biomaterials which could chelate different ions or metals.

### REFERENCE List

101 metal core (Prussian Blue or analogue)
102 metal shell, with bound not core metal or mixed with core metal
103 biocompatible coating (adsorbed proteins or other molecules)
201 metal core (Prussian Blue or analogue)
202 metal shell, with bound not core metal or mixed with core metal
203 biocompatible coating (absorbed proteins or other molecules)
301 metal core (Prussian Blue or analogue)
302 metal shell, with bound not core metal or mixed with core metal
303 biocompatible coating (absorbed proteins or other molecules)
304 metal or ions bound to chelator molecule or protein

### DETAILED DESCRIPTION OF INVENTION

Accordingly, the invention relates to Prussian Blue based nanoparticles comprising a Prussian Blue based metal core doped with one or more metal isotope and an organic biocompatible coating.

The fact that Prussian Blue is indeed ferric ferrocyanide [Fe(III)₄[Fe(II)(CN)₆]₃] with iron(III) atom coordinated to nitrogen and iron(II) atom coordinated to carbon has been definitely established by spectroscopic investigations. Prussian Blue can be synthesized chemically by mixing of ferric (ferrous) and hexacyanoferrate ions with different oxidation state of iron atoms: either Fe³⁺[Fe(II)(CN)₆]⁴⁻ or Fe²⁺[Fe(III)(CN)₆]³⁻. Prussian Blue has a basic cubic structure consisting of alternating iron(II) and iron(III) located on a face centred cubic lattice in such way that the iron(III) ions are surrounded octahedrically by nitrogen atoms, and the iron(II) ions are surrounded by carbon atoms. However the crystals of the Prussian Blue crystals are prone to have defects or vacancies in the crystal lattice. Therefore they may be porous and the vacancies can be occupied by other metal ions. The theoretical structure of Prussian Blue analogue crystal compositions is (PBa) AₓM′ₘ[M(CN)₆]ₙ (see below).

The nanoparticles according to the invention comprise of a metal core, a metal shell (doped with metal isotope(s)) and a biocompatible coating.

Said metal core comprises one or more of Prussian Blue (PB) or Prussian Blue analogue (PBa) of the formula

AₓM′ₘ[M(CN)₆]ₙ

wherein
A denotes a metal selected from the group consisting of Li, Na, K, Rb, Cs, Fr, and Tl,
M denotes a metal selected from the group consisting of V, Cr, Mn, Fe, Co, Ni, Cu, Ga, Zr, Nb, Mo, Ru, Cd, In, Hf, Ta, W, Os and Hg,
M′ denotes a metal selected from the group consisting of Ca, V, Cr, Mn, Fe, Co, Ni, Cu, Zn, Ga, Sr, Zr, Nb, Mo, Ru, Rh, Pd, Ag, Cd, In, Lu, Ba, Hf, Ta, W, Os, Pt, Hg, La, Eu, Gd, Tb, Dy and Ho,
m denotes 0 to 5,
x denotes 0 to 5, and
n denotes 0.5 to 10.

In a preferred embodiment of the invention

A denotes a metal selected from the group consisting of K, Cs, and Tl,
M denotes a metal selected from the group consisting of V, Cr, Mn, Fe, Co, Ni, Cu, Ga, Mo, Ru, In and W,
M′ denotes a metal selected from the group consisting of V, Cr, Mn, Fe, Co, Ni, Cu, Ga, Mo, Ru, Ag, In, W, Pt and Hg,
m denotes 0 to 5,
x denotes 0 to 5, and
n denotes 0.5 to 10.

In a further preferred embodiment of the invention the metal core comprises a mixture of PB and/or one or more PBa as identified above.

In a further preferred embodiment of the invention the PBa is selected from the group consisting of Gd[Fe(CN)₆], KV[Cr(CN)₆], Co[Cr(CN)₆]_{2/3}.

Said metal shell comprises one or more metal isotopes doped to PB or PBa. Said metal isotope is selected from the group consisting of Li, Na, K, Rb, Cs, Fr, Ga, In, Tl, Ca, Sc, V, Cr, Mn, Fe, Co, Ni, Cu, Zn, Sr, Y, Zr, Nb, Mo, Ru, Rh, Pd, Ag, Cd, Ba, La, Sm, Eu, Gd, Tb, Dy, Ho, Lu, Hf, Ta, W, Re, Os, Ir, Pt, Au, Hg, Pb and Bi.

In a preferred embodiment of the invention the metal isotope is selected from the group consisting of Cs, Ga, Tl, In, V, Cr, Mn, Fe, Co, Ni, Cu, Mo, Ru, Ag, W, Pt, Au, Hg, Eu and Gd.

In a further preferred embodiment of the invention the metal shell comprises a mixture of two or more metal isotope as identified above.

Said metal isotope can be present in any sufficient oxidation state theoretically possible. In a further preferred embodiment of the invention the metal isotope is selected from the group consisting of Li(I), Na(I), K(I), Cs(I), Fr(I), Ga(III), In(III), Tl(I), Tl(III), Ca(II), Sc(III), V(III), V(IV), Cr(II), Cr(III), Mn(II), Mn(IV), Fe(II), Fe(III), Co(II), Co(III), Ni(II), Ni(III), Cu(I), Cu(II), Zn(II), Sr(II), Y(III), Zr(IV), Nb(IV), Nb(V), Mo(IV), Mo(V), Ru(III), Ru(IV), Rh(II), Rh(III), Rh(IV), Pd(II), Pd(IV), Ag(I), Cd(II), Ba(II), La(III), Sm(II), Sm(III), Eu(II), Eu(III), Gd(III), Tb(III), Tb(IV), Dy(III), Ho(III), Lu(III), Hf(IV), Ta(V), W(IV), W(V), Re(I), Os(IV), Ir(II), Ir(III), Pt(II), Pt(IV), Au(I), Au(III), Hg(I), Hg(II), Pb(II), Pb(IV) and Bi(III).

Furthermore, said metal isotope can be linked to the PB or PBa by chemical or physical route. In case of a chemical linkage, the metal isotope is bound for example by covalent bond, whereas the metal isotope replaces the Fe atom in the complex structure of PB or PBa. That means with other words the use of a PBa of formula AₓM′ₘ [M(CN)₆]ₙ, wherein M and M′ denote the same or different and independently from each other Cu-61, Cu-64, Cu-67, Zn-62, Zn-69m, Zn-69, Pb-206, Pb-207, Pb-208, Pb-209, Pb-210, Pb-211, Pb-212, Pb-213, Pb-214, Ag-105, Ag-106, Ag-112, Ag-113, Pt-186, Pt-187, Pt-188, Pt-190, Pt-191, Pt-197, La-131, La-132, La-133, La-135, La-140, La-141, La-142, Eu-150m, Eu-152m, Eu-158, Eu-145, Eu-146 and Eu-147, especially Cu-61, Cu-64, Cu-67, Ag-105, Ag-106, Ag-112, Ag-113, Pt-185, Pt-186, Pt-187, Pt-188, Pt-189, Pt-190, Pt-19 and Pt-197. A, m, x and n are as defined above. In a preferred embodiment of the invention the PBa is selected from the group consisting of Ag₄[Fe(CN)₆], Pb₂[Fe(CN)₆], Sn₂[Fe(CN)₆] and Co[Cr(CN)₆]_{2/3}.

In case of a physical linkage, the metal isotope is bounded by physical or physicochemical bonds, such as ion exchange, absorption, mechanical trapping. In this case the metal isotope can be for example adsorbed on the surface of the PB or PBa complex crystals, or it is incorporated into the vacancies.

In a further aspect metal isotope emitting any kind of radiation, such as alpha, beta, gamma or positron radiation can be used. In a preferred embodiment of the invention metal isotope emitting alpha or beta radiation is used, such as Sc-47, Sc-48, Cu-67, Zn-69, Rb-86, Rb-84, Y-90, Zr-95, Zr-97, Nb-95, Nb-96, Nb-98, Ag-112, Ag-113, Cd-115, Cd-117, Cd-118, Cs-136, Cs-138, La-140, La-141, La-142, Sm-153, Eu-150m, Eu-152m, Eu-158, Tb-149, Dy-165, Dy-166, Ho-164, Ho-166, Ho-167, Hf-183, Ta-183, Ta-184, Ta-185, Re-186, Re-188, Re-189, Os-191, Os-193, Os-194, Os-195, Os-196, Ir-193, Ir-195, Pt-197, Pt-200, Au-196, Au-199, Hg-203, Hg-208, Pb-209, Pb-212, Bi-212 and Bi-213.

In another preferred embodiment of the invention metal isotope emitting gamma or positron radiation is used, such as Sc-43, Sc-44, Cu-61, Cu-64, Zn-62, Zn-69m, Ga-67, Ga-68, Rb-81, Rb-82m, Y-84, Y-85, Y-86, Zr-86, Zr-87, Zr-88, Zr-89, Zr-90, Nb-88, Nb-89, Nb-90, Ag-105, Ag-106, Cd-104, Cd-105, Cd-107, Cd-111, Cs-127, Cs-129, Cs-131, Cs-134, Cs-135, La-131, La-132, La-133, La-135, Sm-141, Sm-142, Eu-145, Eu-146, Eu-147, Eu-152m, Tb-147, Tb-150, Tb-151, Tb-152, Tb-154, Tb-154m, Tb-156, Tb-156m, Dy-152, Dy-153, Dy-155, Dy-157, Ho-155, Ho-156, Ho-158, Ho-159, Ho-160, Ho-164, Hf-166, Hf-168, Hf-170, Hf-171, Hf-173, Hf-179, Ta-171, Ta-172, Ta-173, Ta-174, Ta-175, Ta-176, Ta-177, Ta-178, Re-181, Re-182, Re-183, Re-184, Re-186, Re-188, Re-190, Os-180, Os-181, Os-182, Os-183, Ir-183, Ir-184, Ir-185, Ir-186, Ir-187, Ir-188, Ir-189, Ir-190, Pt-185, Pt-186, Pt-187, Pt-188, Pt-189, Pt-190, Pt-191, Pt-197, Au-190, Au-191, Au-192, Au-193, Au-194, Au-196, Au-198, Au-199, Au-200, Au-201, Hg-190, Hg-191, Hg-193, Hg-197, Tl-194, Tl-195, Tl-196, Tl-197, Tl-198, Tl-199, Tl-200, Tl-201, Tl-202, Tl-203, Tl-204, Pb-206, Pb-207, Pb-208, Pb-209, Pb-210, Pb-211, Pb-212, Pb-213, Pb-214, Bi-200, Bi-201, Bi-201, Bi-203, Bi-204, Bi-205 and Bi-206.

Said biocompatible coating comprises biocompatible materials assisting to in vivo and in vitro use of the nano-particles according to the invention. To facilitate cellular delivery and specific intracellular targeting, a probe domain and delivery/targeting ligands must be conjugated to the nanoparticle surface. These sophisticated nanoparticle probes can be multifunctional, including self-delivery into deep tissue, targeting specific cell types, and generating contrast based on target-specific clustering or other molecular switch mechanisms.

Suitable biocompatible materials are any material controlling in vivo and in vitro behaviour, for example natural biological compounds, and natural and synthetic monomers, oligomers and polymers.

In a preferred embodiment of the invention the natural biological compound is selected from the group consisting of cell fragments, cells, bacteria fragments, substances from the large group of lectins, cell surface receptor ligands, hormones and mediator substances, proteins and neoproteins, peptides and polypeptides, antibodies, antibody fragments or the “molecular recognition units” of integrins (ELAM, LECAM, VCAM, etc.) or receptor-specific substances (such as Lewis-X, Sialyl-Lewis-X, etc.), or the great number of blood/plasma/serum components and opsonins, the group of oligonucleotides and synthetic oligonucleotides, DNA and RNA or their derivatives or fragments or analogues (PNA) and homologues, from the group of lipopolysaccharides, lipoproteins, glycerol esters, cholesterols and esters, or metabolites and antimetabolites, cytostatic agents, medical substances, conjugates of medical substances, chemotherapeutical substances and cytostatic agents. Chemical and/or enzymatically produced derivatives or decomposition thereof may be used in addition to, or instead of, the above substances.

In a further preferred embodiment of the invention the natural monomer, oligomer or polymer is selected from the group consisting of natural oligo- and polysaccharides such as dextran with molecular weights of less than 100,000 Da, mixtures of various dextrans, dextrans of different origin, specially purified dextran (FP=free pyrogene quality), fucoidan, arabinogalactan, chondroitin and its sulfates, dermatan, heparin, heparitin, hyaluronic acid, keratan, polygalacturonic acid, polyglucuronic acid, polymannuronic acid, inulin, polylactose, polylactosamine, polyinosinic acid, polysucrose, amylose, amylopectin, glycogen, glucan, nigeran, pullulan, irisin, asparagosin, sinistrin, tricitin, critesin, graminin, sitosin, lichenin, isolichenan, galactan, galactocarolose, luteose, mannans, mannocarolose, pustulan, laminarin, xanthene, xylan and copolymers, araboxylan, arabinogalactan, araban, laevans (fructosans), teichinic acid, blood group polysaccharides, guaran, carubin, alfalfa, glucomannans, galactoglucomannans, phosphomannans, fucans, pectins, cyclodextrins, alginic acid, tragacanth and other gums, chitin, chitosan, agar, furcellaran, carrageen, cellulose, celluronic acid or arabinic acid.

In a further preferred embodiment of the invention the synthetic monomer, oligomer and polymer is selected from the group consisting of polyethylene glycol, polypropylene glycol, polyoxyethylene ether, polyanethol sulfonic acid, polyethylene imine, polymaleimide, polyvinyl alcohol, polyvinyl chloride, polyvinyl acetate, polyvinyl pyrrolidone, polyvinyl sulfate, polyacrylic acid, polymethacrylic acid, polylactide, polylactide glycide. Monosugars to oligosugars and related substances such as aldo- and ketotrioses to aldo- and ketoheptoses, ketooctoses and ketononoses, anhydrosaccharides, monocarboxylic acids and derivatives containing 5 or 6 carbon atoms in the main chain, cyclites, amino and diamino derivatives of saccharides, deoxysaccharides, aminodeoxysaccharides and amino saccharide carboxylic acids, aminocyclites, phosphor-containing derivatives of mono- to oligomers.

In a further preferred embodiment of the invention the biocompatible material is selected from the group consisting of monomer or oligomer carbohydrates or derivatives having antitumoral properties (higher plants, fungi, lichens and bacteria) such as lipopolysaccharides, or containing one or more of the following structures: [3-2,6-fructan, [3-1,3-glucan, mannoglucan, mannan, glucomannan, [3-1,3/1,6-glucans, [3-1,6-glucan, [3-1,3/1,4-glucan, arabinoxylan, hemicellulose, [3-1,4-xylan, arabinoglucan, arabinogalactan, arabinofucoglucan, ot-1,6/1,3-glucan, ot-1,5-arabinan, ot-1,6-glucan, [3-2,1/2,6-fructan, [3-2,1-fructan.

In a further preferred embodiment of the invention the biocompatible material is selected from the group consisting of tensides and surface-active substances such as niotensides, alkyl glucosides, glucamides, alkyl maltosides, mono and polydisperse polyoxyethylene, quaternary ammonium salts, bile acids, alkyl sulfates, betaines, CHAP derivatives.

In a further preferred embodiment of the invention the biocompatible material is selected from the group consisting of antibodies, dextran, polyethylene glycol, polyvinyl pyrrolidone and citrate.

In the nanoparticles according to the invention the amount of the metal core, that of the metal shell and that of the biocompatible coating can be varied in a broad range. In a preferred embodiment of the invention the amounts of the metal core, the metal shell and biocompatible coating are 0.1 to 99% by weight for metal core, 0.0001 to 20% by weight for metal shell and 0.001 to 50% by weight for biocompatible coating, especially 1 to 80% by weight for metal core, 0.0005 to 5% by weight for metal shell and 0.01 to 20% by weight for biocompatible coating, preferably 5 to 70% by weight for metal core, 0.01 to 0.5% by weight for metal shell and 0.1 to 10% by weight for biocompatible coating.

Furthermore, the size of the nanoparticles according to the invention can be varied in a broad range. In a preferred embodiment of the invention the size of the nanoparticles is within the range 1-2000 nm, especially 5-500 nm. In a more preferred embodiment of the invention the size of the nanoparticles is within the range 1-1000 nm, especially 10-350 nm.

As a further aspect the invention relates to a process for the preparation of the nanoparticles according to the invention, wherein a Prussian Blue based metal core is doped with one or more metal isotope, and the doped metal core is coated with one or more biocompatible material.

The process for the preparation of the nanoparticles according to the invention can be performed by usual methods known in the field (for example U.S. Pat. No. 5,928,958, U.S. Pat. No. 7,678,188).

In a preferred method the starting material is PB nanoparticle, which is commercialized or can be synthesized from FeCl₃ and K₄[Fe(CN)₆] which were acidified for example with organic or inorganic acids (such as HCl, citric acid etc.) which is mixed. During mixing the solution is usually temperature and pH controlled because these are crucial parameters in the formation of the nanoparticle determining the core diameter. Additives helping the formation of nanoparticles with homogeneous size distribution and subsequent incorporating the metal isotope and/or covering the nanoparticle with biocompatible coating can be used as well. Then PB or PBa as obtained is mixed in subject or in the form of solution with the salt of the metal isotope or the solution thereof. The last step is adding the biocompatible material to the heavy metal doped PB or PBa.

The nanoparticles according to the invention can be used as subject or in the form of composition comprising the nanoparticles according to the invention as active ingredient beside the usual additives, such as solid or liquid carrier, diluents, etc., known in the field. The form of the composition can be for example tablets, granules, solutions, dispersions, emulsions, etc. The amount of the nanoparticles according to the invention varies between 0.1 to 99% by weight in the composition. Said composition can be prepared by the usual known methods, such as a simple mixing of the nanoparticles according to the invention with the additives.

As the active ingredient of a medicament, two or more types of said nanoparticles may be used in combination.

The dose and frequency of administration of the medicament are not particularly limited, and they may be appropriately chosen depending on conditions such as a purpose of preventive and/or therapeutic treatment, a type of a disease, the body weight or age of a patient, severity of a disease and the like. Generally, a daily dose for oral administration to an adult may be 0.01 to 1,000 mg (the weight of an active ingredient), and the dose may be administered once a day or several times a day as divided portions, or once in several days.

For the use as imaging contrast agent, the route of administration is determined by the path by which labelled nanoparticle is taken into the living human and animal body. Application location: epidural, intacereblar, intracerebroventicular, epicutaneous, intradermal, subcutaneous, nasal, intravenous, intraarterial, intramuscular, intracardiac, intraosseus, intrathecal, introsynovial, intraperitoneal, intravesical, intravitreal, intra-articular, intracavernous, intravaginal, intrauterine, transdermal, transmucosal. The form of these administration are injection (slow or bolus methods) or infusion.

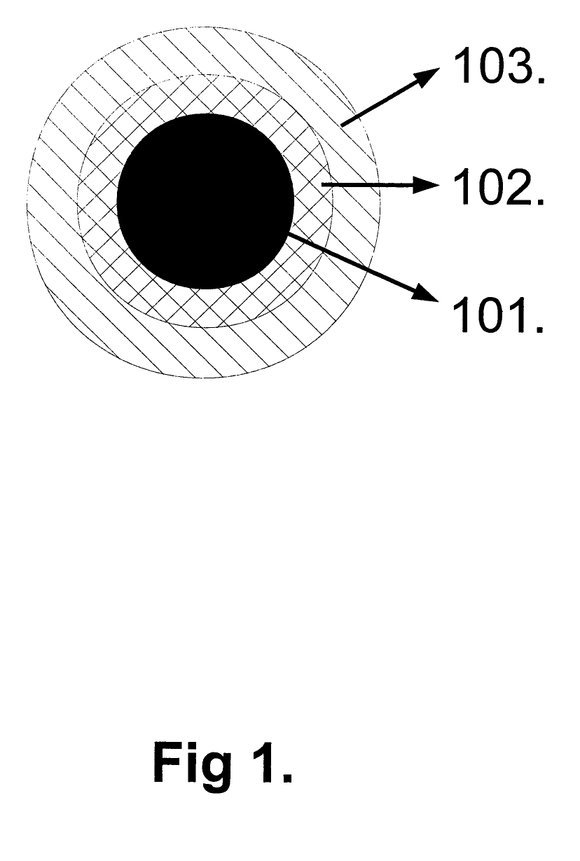

FIG. 1 shows a preferred embodiment of invention. The metal core, such as iron core (101) is made of an iron material selected from the group consisting of Prussian Blue or Prussian Blue analogue defined above. The metal shell (102) is made of different kinds of heavy metals. Prussian Blue FIG. 1 shows an embodiment of the invention, wherein the metal shell and the biocompatible coating compose as continuous layers around the metal core.

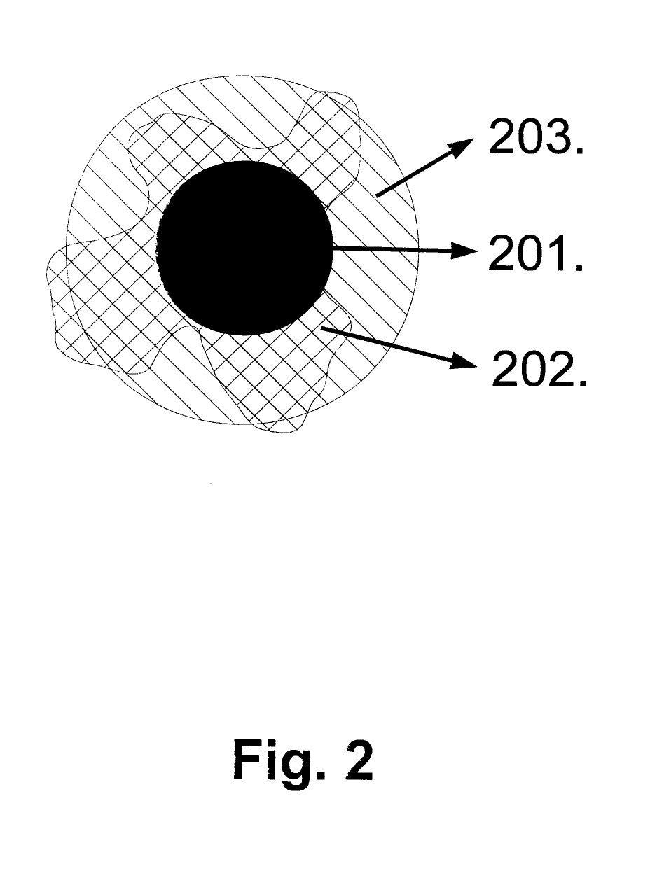

FIG. 2 shows a further preferred embodiment of the invention. The metal shell (202) and biocompatible coating (203) may not be separated clearly. The metal shell does not compose a continuous layer, but is diffused through and over the biocompatible coating. The loosely localized molecules of the biocompatible coating do prevent the diffusion of metal shell added to the surface of the metal core.

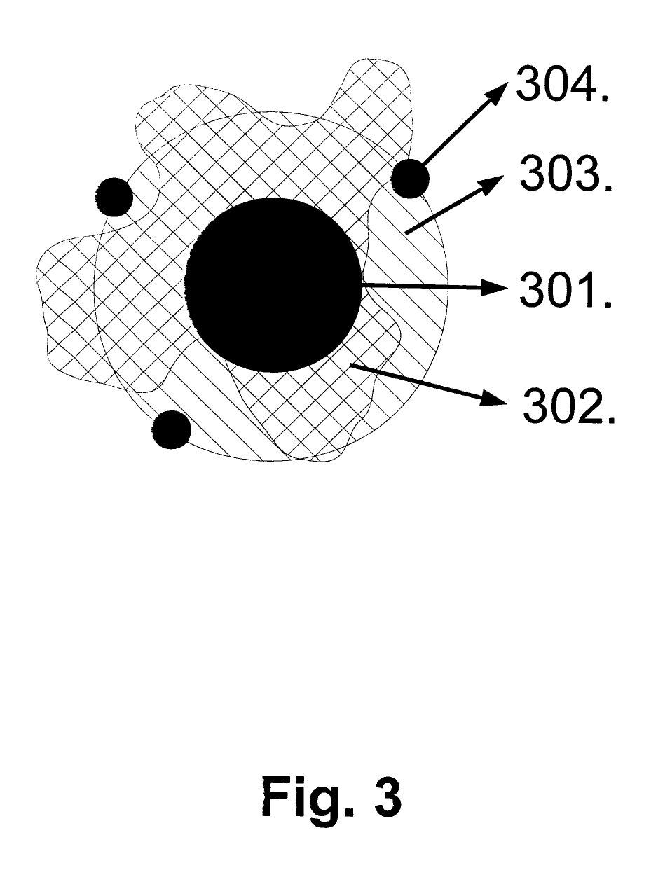

FIG. 3 shows a further preferred embodiment of the invention with the use of dual metal shell, such as isotope and MRI labelling. The biocompatible coating may contain chelator molecules which trap metal or non-metal isotope (304). The metal core (301) and/or metal shell (302) contain different isotopes and/or metals. The dual isotope signalling via local correlation of isotope activity could be applied in functional imaging which helps to find the place of the biodegradation of the isotope or MRI metal chelator molecule.

The nanoparticles according to the invention can be used as a multi modal imaging contrast agent for different imaging methods, such as MRI, CT, SPECT, PET by using nanoparticle according to the invention containing metal isotope emitting gamma or positron radiation, such as Sc-43, Sc-44, Cu-61, Cu-64, Zn-62, Zn-69m, Ga-67, Ga-68, Rb-81, Rb-82m, Y-84, Y-85, Y-86, Zr-86, Zr-87, Zr-88, Zr-89, Zr-90, Nb-88, Nb-89, Nb-90, Ag-105, Ag-106, Cd-104, Cd-105, Cd-107, Cd-111, Cs-127, Cs-129, Cs-131, Cs-134, Cs-135, La-131, La-132, La-133, La-135, Sm-141, Sm-142, Eu-145, Eu-146, Eu-147, Eu-152m, Tb-147, Tb-150, Tb-151, Tb-152, Tb-154, Tb-154m, Tb-156, Tb-156m, Dy-152, Dy-153, Dy-155, Dy-157, Ho-155, Ho-156, Ho-158, Ho-159, Ho-160, Ho-164, Hf-166, Hf-168, Hf-170, Hf-171, Hf-173, Hf-179, Ta-171, Ta-172, Ta-173, Ta-174, Ta-175, Ta-176, Ta-177, Ta-178, Re-181, Re-182, Re-183, Re-184, Re-186, Re-188, Re-190, Os-180, Os-181, Os-182, Os-183, Ir-183, Ir-184, Ir-185, Ir-186, Ir-187, Ir-188, Ir-189, Ir-190, Pt-185, Pt-186, Pt-187, Pt-188, Pt-189, Pt-190, Pt-191, Pt-197, Au-190, Au-191, Au-192, Au-193, Au-194, Au-196, Au-198, Au-199, Au-200, Au-201, Hg-190, Hg-191, Hg-193, Hg-197, Tl-194, Tl-195, Tl-196, Tl-197, Tl-198, Tl-199, Tl-200, Tl-201, Tl-202, Tl-203, Tl-204, Pb-206, Pb-207, Pb-208, Pb-209, Pb-210, Pb-211, Pb-212, Pb-213, Pb-214, Bi-200, Bi-201, Bi-201, Bi-203, Bi-204, Bi-205 and Bi-206.

Single photon emission computed tomography (SPECT, or less commonly, SPET) is a nuclear medicine tomographic imaging method using gamma rays. The basic technique requires injection of a gamma-emitting radioisotope (called radionuclide) into the bloodstream of the patient. In a special case of SPECT, the contrast material of interest for its radioactive properties, is attached to a special radioligand with chemical binding properties to certain types of tissues. This allows the combination of radioisotope with a ligand and bounding the combination to a target within the body, which then (due to the gamma-emission of the isotope) allows the ligand concentration to be shown by a gamma-camera. In the acquired SPECT imaging technique the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. This gives a total scan time of 15-20 minutes. During this image technique an algorithm calculates the 3D reconstruction of local distribution of isotope in the body. In nuclear medicine the characteristic uptake parameter is calculated either pixel-wise or over a region of interest (ROI) for 3D reconstruction as radioactivity or the ratio of radioactivity and body weight and total injected dose (standardised uptake volume). The activity or standardised uptake volume are the more usually used parameter in quantification of in vivo distribution of different labelled drugs, radiopharmacons or labelled nanoparticles.

Positron emission tomography (PET) is a nuclear medicine imaging method, which produces a three-dimensional image or picture of functional processes in the body. The system detects pairs of gamma rays emitted indirectly by a positron-emitting radionuclide (tracer), which is introduced into the body on a biologically active molecule. To conduct the scan, a short half-life radioactive tracer isotope is injected into the subject (usually into blood circulation). The tracer is chemically incorporated into a biologically active molecule. The molecule most commonly used for this purpose is ¹⁸F-fluorodeoxyglucose (FDG), a carbohydrate, for which the waiting period is typically an hour. During the scan a record of tissue concentration is made as the tracer decays. With this imaging technique an algorithm calculates the 3D reconstruction of local distribution of isotope in the body.

The metal composition of the metal core or/and that of the metal shell is the key to detectability of the nanoparticle according to the invention. The isotope, the magnetic and electrical density is the most important determinant of detectability for different imaging methods.

Furthermore, the nanoparticle compound according to the invention can be used for therapeutic purposes by using nanoparticles according to the invention containing metal isotope emitting alpha or beta radiation such as Sc-47, Sc-48, Cu-67, Zn-69, Rb-86, Rb-84, Y-90, Ag-112, Ag-113, Cs-136, Cs-138, La-140, La-141, La-142, Sm-153, Eu-150m, Eu-152m, Eu-158, Tb-149, Dy-165, Dy-166, Ho-164, Ho-166, Ho-167, Re-186, Re-188, Re-189, Ir-193, Ir-195, Pt-197, Pt-200, Au-196, Au-199, Pb-209, Pb-212, Bi-212 and Bi-213.

Said nanoparticles can be used for the therapy and prophylaxis of different diseases and disorders, such as but not excluding other types: oncological diseases most importantly like prostate cancer and metastatic prostate cancer, mammary cancer of all histological types, hepatocellular and biliary route cancers, cancers of the oropharynx and head and neck, cancers arising from the tissue types of thyroid gland, epidermal cancers such as penile, vulvar or skin adenocarcinomas, melanomas and amelanotic melanomas, brain tumors such as astrocytoma, glioblastoma multiforme, metastatic cancers from the lungs (e.g. small cell and non-small cell lung cc.) or from the intestines, neuro-endocrine cancers. Other diseases include sarcoidosis, mycobacterial infections (leprosy, Buruli ulcer, tuberculosis) and infections caused by fungal and protozoal pathogens.

A special case of the use of the nanoparticles according to the invention in the therapy is for example the detection of demage of blood brain barrier (BBB). The main role of BBB is to deffend the brain tissue. Some diseases (such as brain tumor, multiple sclerosis, stroke, meningitis, etc.) induce a damage of BBB, therefore the detection of related part of brain is a clinical evidence. In this example nanoparticles according to the invention, such as ²⁰¹Tl-doped PBa are administered as an intravenous bolus injection and we assume that the input function to the brain is represented by the concentration of PBa in the brachial artery, from which blood is sampled. It is known that PBa is not able to pass through the normal human BBB. To detect the injuries of the BBB, Tc-DTPA and Tc-HMPAO are applied in nuclear medicine. However, these molecules are tiny and they mark the injured area as a positive or negative contrast. PB can be used for diagnostics primarily as an indicator of the injuries of the blood-brain-barrier. The BBB is a subject of active research being examined from several approaches. Generally speaking, researchers try to target nanoparticles used for the purposes of both therapy and diagnostics by surface functionalisation, thus getting them through the ISO 17021 (US2004204354A1, US2004204354A1, WO2009136763A2). Particles of various size may get into the brain tissue in different quantities at the location of the injury. Thus, scalable nanoparticles may facilitate the estimation of the rate of BBB injuries, providing for the appropriate diagnostic and therapeutic effect in the targeted area.

As a summary of the information described above, the metal isotope suitable for the metal shell of the nanoparticles according to invention are listed in the following table indicating the kind of the radiation emitted by said isotope, which is determinant for the use in the imaging method or therapy.

| | | e+ | g | e− | a |
|---|---|---|---|---|---|
| Scandium (Z = 21) | Sc | x | x | x | |
| Copper (Z = 29) | Cu | x | x | x | |
| Zinc (Z = 30) | Zn | x | x | x | |
| Rubidium (Z = 37) | Rb | x | x | x | |
| Yttrium (Z = 39) | Y | x | x | x | |
| Zirconium (Z = 40) | Zr | x | x | x | |
| Niobium (Z = 41) | Nb | x | x | x | |
| Silver (Z = 47) | Ag | x | x | x | |
| Cadmium (Z = 48) | Cd | x | x | x | |
| Caesium (Z = 55) | Cs | x | x | x | |
| Lanthanum (Z = 57) | La | x | x | x | |
| Samarium (Z = 62) | Sm | x | x | x | x |
| Europium (Z = 63) | Eu | x | x | x | |
| Terbium (Z = 65) | Tb | x | x | x | x |
| Dysprosium (Z = 66) | Dy | x | x | x | x |
| Holmium (Z = 67) | Ho | x | x | x | x |
| Hafnium (Z = 72) | Hf | x | x | x | |
| Tantalum (Z = 73) | Ta | x | x | x | |
| Rhenium (Z = 75) | Re | x | x | x | |
| Osmium (Z = 76) | Os | x | x | x | |
| Iridium (Z = 77) | Ir | x | x | x | |
| Platinum (Z = 78) | Pt | x | x | x | |
| Gold (Z = 79) | Au | x | x | x | |
| Mercury (Z = 80) | Hg | x | x | x | |
| Thallium (Z = 81) | Tl | x | x | x | |
| Lead (Z = 82) | Pb | x | x | x | x |
| Bismuth (Z = 83) | Bi | x | x | x | x |
| Sodium (Z = 11) | Na | x | | x | |
| Potassium (Z = 19) | K | x | | x | |
| Calcium (Z = 20) | Ca | | | x | |
| Vanadium (Z = 23) | V | x | x | x | |
| Chromium (Z = 24) | Cr | x | x | x | |
| Manganese (Z = 25) | Mn | x | x | x | |
| Iron (Z = 26) | Fe | x | x | x | |
| Cobalt (Z = 27) | Co | x | x | x | |
| Nickel (Z = 28) | Ni | x | | x | |
| Molybdenum (Z = 42) | Mo | x | x | x | |
| Ruthenium (Z = 44) | Ru | x | x | x | |
| Rhodium (Z = 45) | Rh | x | x | x | |
| Palladium (Z = 46) | Pd | x | x | x | |
| Indium (Z = 49) | In | x | x | x | |
| Barium (Z = 56) | Ba | x | x | x | |
| Gadolinium (Z = 64) | Gd | x | x | x | |
| Lutetium (Z = 71) | Lu | x | x | x | |
| Tungsten (Z = 74) | W | x | x | x | |
| Francium (Z = 87) | Fr | x | x | x | x |

g: gamma radiation; e−: beta radiation; a: alpha radiation; e+: positron radiation

Accordingly, as a further aspect the invention relates to Prussian Blue based nanoparticles according to the invention for use as imaging contrast material.

Furthermore, the invention relates to Prussian Blue based nanoparticles according to the invention for use in therapy.

Furthermore, the invention relates to use of the Prussian Blue based nanoparticles according to the invention as imaging contrast material.

Furthermore, the invention relates to use of the Prussian Blue based nanoparticles according to the invention in therapy.

The invention is described in more detail with the following non-limiting examples:

### Example 1

PB with Citric Acid (PB_{—}2010Fe_AC)

Solution A contains 3.25 mg (0.02 mmol) FeCl₃ and 96 mg (0.5 mmol) citric acid in 15 ml double distilled water. Solution B contains 8.44 mg (0.02 mmol) K₄[Fe(CN)₆]×3H₂O and 96 mg (0.5 mmol) citric acid in 20 ml double distilled H₂O at room temperature. (pH_{A}=2.40; pH_{B}=2.65). The solutions A and B were mixed at room temperature. Heated to 50° C. by water bath and mixed during 15 minutes. The next step of procedure is spinned for 30 min at 30000/min at 4° C. The volume of produced nanoparticles was determined precisely with dynamic light scattering and the diameter was 40 nm. The absorption (UV and Vis) and IR spectrum of nanoparticle are tipical of size and material therefore the standardisation of different product was made by absorption and IR spectrophotometry. The UV-vis spectrum of the resulting solution showed a broad band at 690 nm. The IR spectra of the prussian blue nanoparticles exhibited a strong peak at 2078 cm-1 (C═N stretching vibration), 1712 and 1364 cm-1 (asymmetric and symmetric carboxyl stretching bands). Based on these results concluded the core was Fe₄[Fe(CN)₆]₃ and biocompatible coating was citric acid 5.2% of total mass of nanoparticle in final product.

### Example 2

### PB with Polyvinylpropylene. (PB_{—}2010Fe_K30)

A solution of 375 mg Kollidon 30 (polyvinyl-pyrolidon) was made in 4.5 ml double distilled H₂O at room temperature, and shaked until obtaining a clear solution. Then the solution was adjusted to pH 2.00 whit 0.5N HCl (4 drops). After then 11 mg K₃[Fe(CN)₆] was washed into it with 0.5 ml double distilled water. After 5 minute mixing the solution was heated in an autoclave for 2 hours at 80° C. After cooled down to room temperature it was spinned for 30 min at 29000/min at 4° C. (Type 50.2 Ti). The volume of the produced nanoparticles was determined precisely with dynamic light scattering and the diameter was 160 nm. The quantification methods see above Example 1. The K30 adsorption to surface of nanoparticle was tested by IR spectroscopy (1650-1680 1/cm). Based on these results concluded the core was Fe₄[Fe(CN)₆]₃ and biocompatible coating was 22.5% of total mass of nanoparticles in final product.

### Example 3

### PB without Biocompatible Corona (PB_{—}2010Fe_Null)

Solution A contains 3.25 mg (0.02 mmol) FeCl₃ and 96 mg in 15 ml double distilled water. Solution B contains 8.44 mg (0.02 mmol) K₄[Fe(CN)₆]ₓ₃H₂O in 20 ml double distilled H₂O at room temperature. The pH are changed in Solution A and B with 5-10 microL cc. Hcl, (pH_{A}=2.02; pH_{B}=2.05). The solutions A and B were mixed at room temperature. Heated to 60° C. by water bath and mixed during 15 minutes. The next step of procedure is spinned for 30 min at 30000/min at 4° C. After sedimentation the nanoparticle was unloaded. The Fe concentration in bulk solution was measured by absorption spectroscopy. The volume of produced nanoparticles was determined precisely with dinamic light scattering and the diameter was 54+/−7 nm. The quantification methods see above. Based on these results concluded the core was Fe₄[Fe(CN)₆]₃.

### Example 4

The example presents an animal (mouse) experiment with model damage of BBB. High-speed microdrill (Dremel) with a diamond point (1 mm) was applied to prepare cranotomy in mice. Cold lesion stamps custom were used made from brass and nickel coated to prevent sticking to the tissue. The stamp itself is a small cup with a pointed bottom and a circular footprint of 3 mm in diameter. (Brain Research Protocols Volume 11, Issue 3, July 2003, Pages 145-154, Pflügers Archiv European Journal of Physiology Volume 440, Number 2, 309-314, DOI: 10.1007/s004240000293). Stamps were cooled in liquid nitrogen to equilibrium and then applied during 60 seconds through the drill hole to the brain surface of mice.

The PB (PB_{—}2010Fe_Null) was added intravenously (0.1 ml, 125 micromol/liter) 5 hours after the cold lesion. The mouse was imaged with the multi-pinhole NanoSPECT/CT camera (Mediso, Hungary). Mouses were anaesthetized with isoflurane/O2. Multi-pinhole apertures with a diameter of 2.5 mm were used on each head, with a field of view (FOV) of 24 mm were applied. Settings of ²⁰¹Tl energy peaks were recorded at the detection heads. Based on previous CT topogram scans, a body range from neck to bottom was scanned in 24 min with 60 s per projection. A 6 min CT at 45 kVp was acquired. Animals were imaged at 2 h measure the after the PB injection allow calculation of the brain tissue and lesion calculation.

### Example 5

MRI study in Eppendorf was carried out on a whole-body animal MR scanner (Mediso, Hungary) operating at 1.5 T. The MR T1 and T2 sequence used to follow Gd-DTPA enhancement in phantom. It consists of 3 Eppendorf tubes of PB (PB_{—}2010Fe_AC) in different concentrations (10 μM, 100 μM, 1000 μM). The above-mentioned spin-echo sequence was applied together. The temperature was 294 K.

### Example 6

We produce new Co containing PB analogues (PB_{—}2010Co_AC) with this method. Solution A contains 4.75 mg CoCl₂×6H₂0 and 98 mg citric acid in 20 ml double distilled water. Solution B contains 8.44 mg (0.02 mmol) K₄[Fe(CN)₆]×3H₂O and 98 mg citric acid in 20 ml double distilled H₂O at room temperature. (pH_{A}=2.40; pH_{B}=2.65). The solutions A and B were mixed at different temperature (25, 40, 60, 80° C.) during 15 minutes. The next step of procedure, which separate nanoparticles from rest part of solution, is spinned for 30 min at 30,000/min at 4° C. The volume of produced nanoparticles was determined precisely with dynamic light scattering and atomic force microscopy (AFM) the diameter was 50-70 nm range depending on temperature of mixing.

| T (C.) | 25 | 40 | 60 | 80 |
|---|---|---|---|---|
| R (nm) | 10.4 +/− 3 | 27.3 +/− 5 | 21.7 +/− 5 | 54.1 +/− 8 |

The PB_{—}2010Co_AC nanoparticles were labelled with different isotopes. These isotopes were 201-Tl (physical form: TlCl, activity 22 MBq), 111-In (physical form: InCl3, activity 32 MBq), 65-Zn (physical form: ZnCl2, activity 1.5 MBq), 52-Mn (physical form: MnCl2, activity 3.4 MBq), 48-V (physical form: VO2, activity 4 MBq). All types of isotopes were solubilized in 1 ml saline. The binding effectivity approximately 75% of these isotopes was tested with thin layer chromatography to PB_{—}2010Co_AC form. The quantification methods see above. The quantification methods see above. Based on these results concluded the core Co₃[Fe(CN)₆]₂ with biocompatible citric acid (6.3%) coating.

### Example 7

The PB_{—}2010Fe_AC nanoparticles were labelled with different isotopes. These isotopes were 201-Tl (physical form: TlCl, activity 22 MBq), 111-In (physical form: InCl3, activity 32 MBq), 65-Zn (physical form: ZnCl2, activity 1.5 MBq), 52-Mn (physical form: MnCl12, activity 3.4 MBq), 48-V (physical form: VO2, activity 4 MBq). All types of isotopes were solubilized in 1 ml saline. The binding effectivity of these isotopes were tested with thin layer chromatography and these were >85% (RF=0.02). During chemical identification (methods see above) we could not measure mass of 201-Tl or 111-In below error range. The structure of nanoparticle core was Fe₄[Fe(CN)₆]₃ and biocompatible coating was citric acid 4.9%.

### Example 8

The 201-Tl labelled citrate coated PB (PB_{—}2010Fe_AC) was added intravenously (0.1 ml, 125 micromol/liter). The bulk solution intergradients were 8.5 MBq 201-Tl in TlCl form, 2.3 mg PB_{—}2010Fe_AC nanoparticles, 8.5 mg citric acid and 9.2 mg NaCl in 1 ml. The aim of this measurement was to determine the biodistribution and time dependence of nanoparticle uptake. The mouse was imaged with the multi-pinhole NanoSPECT/CT camera (Mediso, Hungary). Mouses were anaesthetized with isoflurane/O2. Settings of ²⁰¹Tl energy peaks were recorded at the detection heads. Based on previous CT topogram scans, a body range from neck to bottom was scanned in 24 min with 60 s per projection. A 6 min CT at 45 kVp was acquired. Animals were imaged at 2 h measure the after the injection. The detected activity (quantification with NanoSPECT/CT) in brain/kidney/liver are 0.39%/16.72%/30.26% of the activity of total body.

### Example 9

The 201-Tl labelled K30 coated PB (PB_{—}2010Fe_K30) was added intravenously (0.1 ml, 125 micromol/liter). The bulk solution intergradients were 7.6 MBq 201-Tl in TlCl form, 2.0 mg PB_{—}2010Fe_AC nanoparticles, 0.2 ul 0.4 N HCl, 0.23 mg glucose and 9.2 mg NaCl in 1 ml. The aim of this measurement was to determine the biodistribution and time dependence of nanoparticle uptake. The mouse was imaged with the multi-pinhole NanoSPECT/CT camera (Mediso, Hungary). Mouses were anaesthetized with isoflurane/O2. Settings of 201Tl energy peaks were recorded at the detection heads. Based on previous CT topogram scans, a body range from neck to bottom was scanned in 24 min with 60 s per projection. A 6 min CT at 45 kVp was acquired. Animals were imaged at 2 h measure the after the injection. The detected activity (quantification with NanoSPECT/CT) in brain/kidney/liver are 0.48%/27.64%/14.64% of the total body activity.

### Example 10

The PB_{—}2010Fe_AC nanoparticles (3.5 mg) was labelled with 65-Zn (physical form: ZnCl2, activity 1.3 MBq) in 1.5 ml saline which contains 13.7 mg NaCl. The solution was placed to syringe and was imaged with the NanoScan (PET/MRI) camera (Mediso, Hungary) during 120 min. After 65-Zn correction calibration the labelled PB_{—}2010Fe_AC was added intravenously (0.1 ml) to mouse (23 g, Wistar) via tain vein which was anaesthetized with isoflurane/O2. The measure was started 95 min after the injection. The detected reconstructed activity in kidney and liver are 22.62% and 13.91% of the total body activity.

### Example 11

The aims of invented PB nanoparticle is the use thereof in clinical and animal in vivo imaging instruments. The next examples introduce a bulk solution which applicable injection or infusion. The A solution intergradients are 2.0 mg PB_{—}2010Fe_AC nanoparticles, 0.2 ul 0.4 N HCl, 0.23 mg glucose and 9.2 mg NaCl in 0.8 ml distilled water as a isotonic solution. The B solution contains 8.6 MBq 201-Tl in TlCl form in 0.2 ml. The A and B solution are prepared and before inject (injection via vein) to body have to be mixed.

The description above contains many specifications. These should not be construed as limiting the scope of the embodiments, but as merely providing illustrations of some of the presently preferred embodiments.

1. A Prussian Blue based nanoparticle comprising a Prussian Blue based metal core doped with one or more metal isotope and an organic biocompatible coating.

2. A Prussian Blue based nanoparticle according to claim 1, wherein said metal core comprises Prussian Blue or one or more Prussian Blue analogue of the formula
AₓM′ₘ[M(CN)₆]ₙ
wherein
A denotes a metal selected from the group consisting of Li, Na, K, Rb, Cs, Fr and Tl,
M denotes a metal selected from the group consisting of V, Cr, Mn, Fe, Co, Ni, Cu, Ga, Zr, Nb, Mo, Ru, Cd, In, Hf, Ta, W, Os and Hg,
M′ denotes a metal selected from the group consisting of Ca, V, Cr, Mn, Fe, Co, Ni, Cu, Zn, Ga, Sr, Zr, Nb, Mo, Ru, Rh, Pd, Ag, Cd, In, Lu, Ba, Hf, Ta, W, Os, Pt, Hg, La, Eu, Gd, Tb, Dy and Ho,
m denotes 0 to 5,
x denotes 0 to 5, and
n denotes 0.5 to 10.

3. A Prussian Blue based nanoparticle according to claim 1, wherein said metal isotope is selected from the group consisting of Li, Na, K, Rb, Cs, Fr, Ga, In, Tl, Ca, Sc, V, Cr, Mn, Fe, Co, Ni, Cu, Zn, Sr, Y, Zr, Nb, Mo, Ru, Rh, Pd, Ag, Ba, La, Stn, Eu, Gd, Tb, Dy, Ho, Lu, Hf, Ta, W, Re, Os, Ir, Pt, Au, Hg, Pb and Bi.

4. A Prussian Blue based nanoparticle according to claim 1, wherein said biocompatible coating comprises one or more biocompatible material selected from the group consisting of antibodies, dextran, polyethylene glycol, polyvinyl pyrrolidone and citrate.

5. A process for the preparation of Prussian Blue based nanoparticles according to claim 1, wherein a Prussian Blue based metal core is doped with one or more metal isotope, and the doped metal core is coated with one or more biocompatible material.

6. An imaging contrast material comprising the Prussian Blue based nanoparticle according to claim 1.

7. A Prussian Blue based nanoparticle according to claim 1 for use in therapy.

8. An tomographic imaging method comprising introducing the Prussian Blue based nanoparticle according to claim 1 into a subject.

9. A therapeutic or diagnostic method comprising introducing the Prussian Blue based nanoparticle according to claim 1 into a subject.

10. A Prussian Blue based nanoparticle according to claim 2, wherein said metal isotope is selected from the group consisting of Li, Na, K, Rb, Cs, Fr, Ga, In, Tl, Ca, Sc, V, Cr, Mn, Fe, Co, Ni, Cu, Zn, Sr, Y, Zr, Nb, Mo, Ru, Rh, Pd, Ag, Ba, La, Stn, Eu, Gd, Tb, Dy, Ho, Lu, Hf, Ta, W, Re, Os, Ir, Pt, Au, Hg, Pb and Bi.

11. A Prussian Blue based nanoparticle according to claim 2, wherein said biocompatible coating comprises one or more biocompatible material selected from the group consisting of antibodies, dextran, polyethylene glycol, polyvinyl pyrrolidone and citrate.

12. A process for the preparation of Prussian Blue based nanoparticles according to claim 2, wherein a Prussian Blue based metal core is doped with one or more metal isotope, and the doped metal core is coated with one or more biocompatible material.
